(19) United States
(12) Patent Application Publication
(10) Pub. No.: US 2024/0065835 A1
(43) Pub. Date: Feb. 29, 2024

(54) SYSTEM FOR DELIVERING A REPLACEMENT HEART VALVE IMPLANT

(71) Applicant: BOSTON SCIENTIFIC SCIMED, INC., MAPLE GROVE, MN (US)

(72) Inventors: Tim O'Connor, Galway (IE); Chris Cullen, Galway (IE)

(73) Assignee: BOSTON SCIENTIFIC SCIMED, INC., MAPLE GROVE, MN (US)

(21) Appl. No.: 18/239,278

(22) Filed: Aug. 29, 2023

Related U.S. Application Data
(60) Provisional application No. 63/402,231, filed on Aug. 30, 2022.

(51) Int. Cl. A61F 2/24

(52) U.S. Cl. CPC A61F 2/2436

(57) ABSTRACT

A system for delivering a replacement heart valve implant may include a handle and a valve capsule, an inner shaft extending from the handle to the capsule, an outer sheath over the inner shaft and extending from the handle to the capsule, a positioning sheath over the outer sheath and extending from the handle, a guide tube within the handle, and a distal hub attached to the positioning sheath. The distal hub may be disposed within and movable relative to the guide tube via rotation of the positioning sheath. A method of manufacturing the system may include positioning proximal and distal hubs within the guide tube, setting a first predetermined distance between proximal and distal capsule portions, moving the positioning sheath relative to the outer sheath to set a second predetermined distance between the hubs, and securing the distal hub within the guide tube at the second predetermined distance.

### CROSS-REFERENCE TO RELATED APPLICATIONS

This application claims the benefit of priority of U.S. Provisional Application No. 63/402,231 filed Aug. 30, 2022, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a system for delivering a replacement heart valve implant and/or methods of manufacturing a system for delivering a replacement heart valve implant.

### BACKGROUND

A wide variety of intracorporeal medical devices have been developed for medical use, for example, intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, a system for delivering a replacement heart valve implant may comprise a proximal handle and a valve capsule spaced apart from the proximal handle, the valve capsule being configured to receive the replacement heart valve implant; an inner shaft extending distally from the proximal handle to the valve capsule; an outer sheath coaxially disposed over the inner shaft and extending distally from the proximal handle to the valve capsule; a positioning sheath coaxially disposed over the outer sheath and extending distally from the proximal handle to a distal end spaced apart proximally from the valve capsule; a guide tube disposed within the proximal handle; and a distal hub fixedly attached to a proximal end of the positioning sheath, the distal hub being disposed within and selectively movable relative to the guide tube via rotation of the positioning sheath.

In addition or alternatively to any example described herein, the distal hub includes a body portion and a helical ridge extending radially outward from the body portion.

In addition or alternatively to any example described herein, the guide tube includes at least one set screw threadably engaged with a wall of the guide tube and configured to extend between adjacent turns of the helical ridge.

In addition or alternatively to any example described herein, the at least one set screw includes two or more set screws.

In addition or alternatively to any example described herein, the system may further comprise a proximal hub fixedly attached to a proximal end of the outer sheath, the proximal hub being disposed within and selectively movable axially relative to the guide tube.

In addition or alternatively to any example described herein, the proximal handle includes a first collar rotatably disposed around the guide tube. Rotation of the first collar about the guide tube may be configured to move the proximal hub axially within the guide tube.

In addition or alternatively to any example described herein, the distal hub acts as a hard stop for axial movement of the proximal hub in a distal direction.

In addition or alternatively to any example described herein, the valve capsule includes a proximal capsule portion fixedly attached to a distal portion of the outer sheath and a distal capsule portion fixedly attached to a distal portion of the inner shaft.

In addition or alternatively to any example described herein, the proximal capsule portion is configured to cover a first portion of the replacement heart valve implant and the distal capsule portion is configured to cover a second portion of the replacement heart valve implant for percutaneous delivery of the replacement heart valve implant to a treatment site.

In addition or alternatively to any example described herein, a method of manufacturing a system for delivering a replacement heart valve implant may comprise:
positioning a proximal hub and a distal hub within a guide tube of a proximal handle of the system, wherein:
   an inner shaft extends through the guide tube to a distal capsule portion of a valve capsule spaced apart from the proximal handle, the valve capsule being configured to receive the replacement heart valve implant;
   the proximal hub is fixedly attached to a proximal end of an outer sheath coaxially disposed over the inner shaft and extending distally from the proximal handle to a proximal capsule portion of the valve capsule;
   the distal hub is fixedly attached to a proximal end of a positioning sheath coaxially disposed over the outer sheath and extending distally from the proximal handle to a distal end spaced apart proximally from the valve capsule; and at least one set screw is threadably engaged with a wall of the guide tube;
setting a first predetermined distance between the proximal capsule portion and the distal capsule portion;
axially moving the positioning sheath relative to the outer sheath to set a second predetermined distance between the proximal hub and the distal hub; and
engaging the at least one set screw with a body portion of the distal hub to secure the distal hub axially within the guide tube at the second predetermined distance from the proximal hub.

In addition or alternatively to any example described herein, axially moving the positioning sheath relative to the outer sheath includes rotating the positioning sheath relative to the outer sheath.

In addition or alternatively to any example described herein, the distal hub includes a helical ridge extending radially outward from the body portion and the at least one set screw extends between adjacent turns of the helical ridge.

In addition or alternatively to any example described herein, engagement of the at least one set screw with the body portion of the distal hub prevents axial movement of the distal hub relative to the guide tube.

In addition or alternatively to any example described herein, the distal hub includes a proximal flange extending radially outward from the body portion farther than the helical ridge and a distal flange extending radially outward from the body portion farther than the helical ridge.

In addition or alternatively to any example described herein, the second predetermined distance is less than the first predetermined distance.

In addition or alternatively to any example described herein, a system for delivering a replacement heart valve implant configured to shift between a collapsed configuration and an expanded configuration may comprise a proximal handle and a valve capsule spaced apart from the proximal handle, the valve capsule being configured to receive and retain the replacement heart valve implant in the collapsed configuration; an inner shaft extending distally from the proximal handle to the valve capsule, wherein the inner shaft extends axially through the replacement heart valve implant when the replacement heart valve implant is disposed within the valve capsule; an outer sheath coaxially disposed over the inner shaft and extending distally from the proximal handle to the valve capsule; a positioning sheath coaxially disposed over the outer sheath and extending distally from the proximal handle to a distal end spaced apart proximally from the valve capsule; a guide tube disposed within the proximal handle; and a distal hub fixedly attached to a proximal end of the positioning sheath and disposed coaxially over the inner shaft, the distal hub being disposed within and selectively movable axially relative to the guide tube.

In addition or alternatively to any example described herein, the guide tube is formed from a metallic material.

In addition or alternatively to any example described herein, the distal hub is formed from a polymeric material.

In addition or alternatively to any example described herein, the distal hub includes a body portion and a helical ridge extending radially outward from the body portion. The guide tube includes at least one set screw threadably engaged with a wall of the guide tube and extending radially inward therefrom. When the at least one set screw extends between adjacent turns of the helical ridge: rotation of the positioning sheath moves the distal hub axially relative to the guide tube when the at least one set screw is disengaged from the body portion of the distal hub; and mechanical interference between the at least one set screw and the helical ridge prevents axial movement of the distal hub relative to the guide tube when an axial force is applied to the distal hub.

In addition or alternatively to any example described herein, the system may further comprise a proximal hub fixedly attached to a proximal end of the outer sheath, the proximal hub being disposed within and selectively movable axially relative to the guide tube. Distal movement of the proximal hub within the guide tube brings the proximal hub into contact with the distal hub and applies the axial force to the distal hub in a distal direction.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a system for delivering a replacement heart valve implant;

FIG. 2 is a partial cross-sectional view illustrating selected aspects of the system of FIG. 1 in a delivery configuration;

FIG. 3 is a detailed view illustrating selected aspects of the system of FIG. 2;

FIG. 4 is a partial cross-sectional view illustrating selected aspects of the system of FIGS. 1-3 in a deployment configuration;

FIG. 5 is a partial cross-sectional view illustrating selected aspects of a handle of the system of FIGS. 1-4;

FIG. 6 is a partial cutaway view illustrating selected aspects of the handle of the system of FIGS. 1-5;

FIGS. 7-8 are partial cross-sectional views illustrating selected aspects of the system of FIGS. 1-6; and

FIG. 9 is a partial cross-sectional view illustrating selected aspects of the system of FIGS. 1-8 in a withdrawal configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device or system. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein are systems and/or methods that may be used in a portion of the cardiovascular system in order to diagnose, treat, and/or repair the system. In some embodiments, the systems and/or methods disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The systems and/or methods disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. As such, it will be understood that the following discussion may apply equally to any and/or all of the components for which there are more than one within the systems and/or methods unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the systems and/or methods disclosed herein may have applications and uses in other portions of a patient's anatomy, such as but not limited to, arteries, veins, and/or other body lumens.

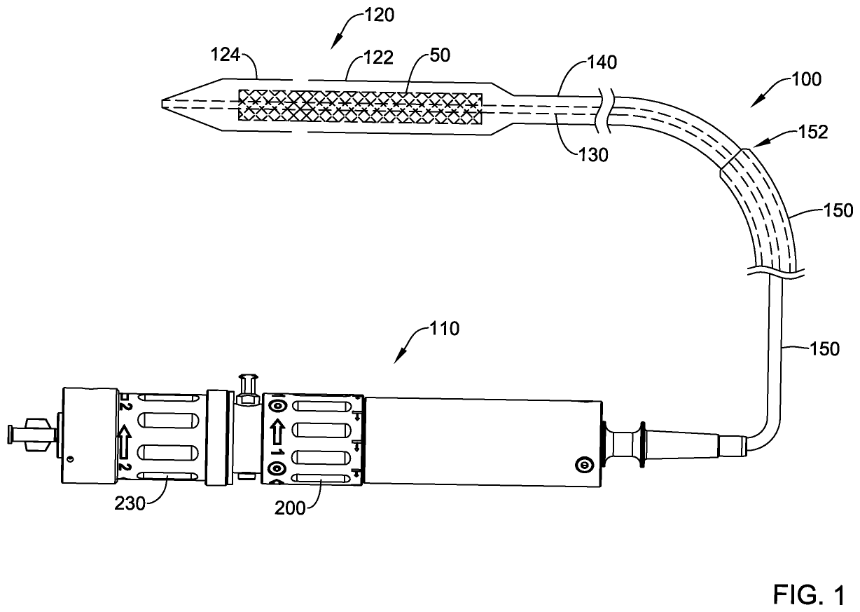

FIG. 1 illustrates selected aspects of a system 100 for delivering a replacement heart valve implant 50 to a treatment site. The replacement heart valve implant 50 is illustrated schematically. The replacement heart valve implant 50 may include an expandable framework defining a central lumen which, in some embodiments, may be substantially cylindrical. In some embodiments, the expandable framework may have a substantially circular cross-section. In some embodiments, the expandable framework can have a non-circular (e.g., D-shaped, elliptical, etc.) cross-section. In some embodiments, a non-circular expandable framework can be used to repair a mitral valve or another non-circular valve in the patient's heart or body. Some suitable but non-limiting examples of materials that may be used to form the expandable framework, including but not limited to metals and metal alloys, composites, ceramics, polymers, and the like, are described below.

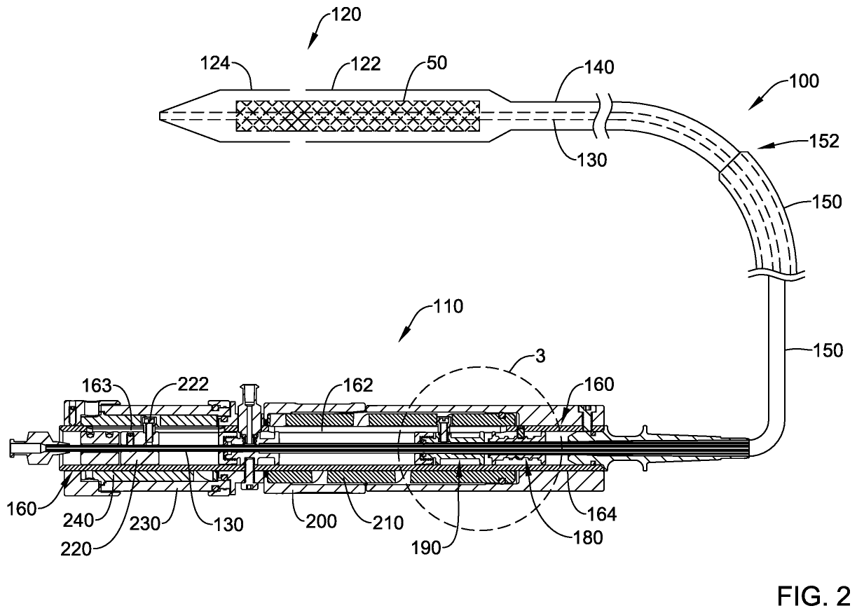

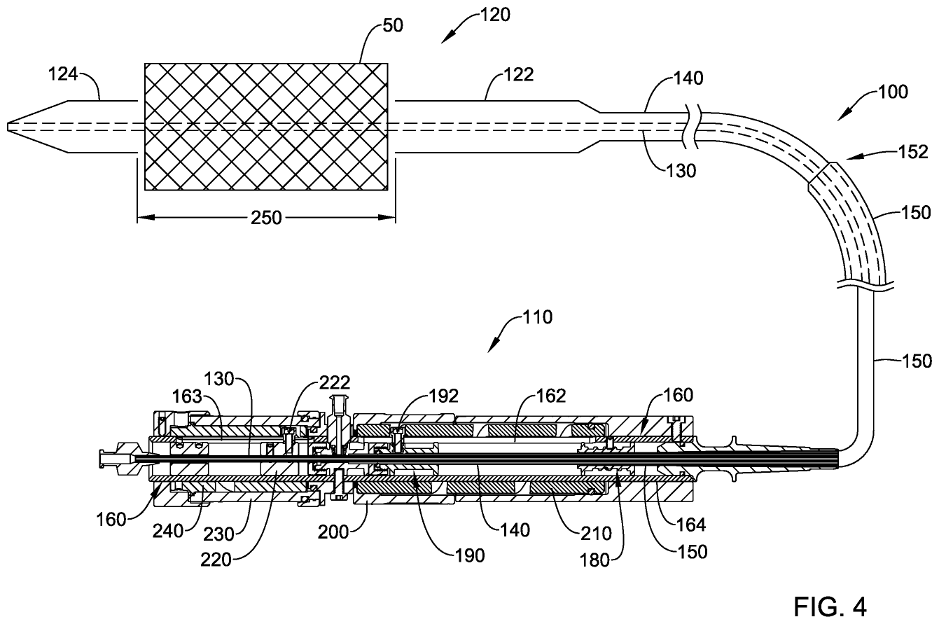

The replacement heart valve implant 50 and/or the expandable framework may be configured to shift between a collapsed configuration (e.g., FIG. 2) and an expanded configuration (e.g., FIG. 4). In some embodiments, the collapsed configuration may be a radially collapsed configuration and the expanded configuration may be a radially expanded configuration. In some embodiments, the expandable framework may be self-expanding. In some embodiments, the expandable framework may be self-biased toward the expanded configuration. In some embodiments, the expandable framework may be mechanically expandable. In some embodiments, the expandable framework may be balloon expandable. Other configurations are also contemplated.

It will be appreciated that the replacement heart valve implant 50 can be any type of heart valve (e.g., a mitral valve, an aortic valve, etc.). The replacement heart valve implant 50 may be configured to allow one-way flow through the replacement heart valve implant 50 from an inflow end to an outflow end. In some embodiments of the replacement heart valve implant 50, the expandable framework may define a lower crown proximate an inflow end of the replacement heart valve implant 50, an upper crown proximate an outflow end of the replacement heart valve implant 50, and a plurality of stabilization arches extending downstream from the outflow end.

In some embodiments, the replacement heart valve implant 50 may include a plurality of valve leaflets disposed within the central lumen. The plurality of valve leaflets may be coupled, secured, and/or fixedly attached to the expandable framework. In some embodiments, the plurality of valve leaflets can be integrally formed with each other, such that the plurality of valve leaflets is formed as a single unitary and/or monolithic unit. In some embodiments, the plurality of valve leaflets may be formed integrally with other structures such as an inner skirt and/or an outer skirt, base structures, liners, or the like. The plurality of valve leaflets may be configured to substantially restrict fluid from flowing through the replacement heart valve implant 50 in a closed position. For example, in some embodiments, free edges of the plurality of valve leaflets may move into coaptation with one another in the closed position to substantially restrict fluid from flowing through the replacement heart valve implant 50. The free edges of the plurality of valve leaflets may move apart from each other in an open position to permit fluid flow through the replacement heart valve implant 50.

In some embodiments, the replacement heart valve implant 50 may include an inner skirt. The inner skirt may be disposed on and/or extend along an inner surface of the expandable framework. In at least some embodiments, the inner skirt may be fixedly attached to the expandable framework. The inner skirt may direct fluid, such as blood, flowing through the replacement heart valve implant 50 toward the plurality of valve leaflets. In at least some embodiments, the inner skirt may be fixedly attached to and/or integrally formed with the plurality of valve leaflets. The inner skirt may ensure the fluid flows through the central lumen and does not flow around the plurality of valve leaflets when they are in the closed position.

In some embodiments, the replacement heart valve implant 50 may include an outer skirt. In some embodiments, the outer skirt may be disposed on and/or extend along an outer surface of the expandable framework. In some embodiments, the outer skirt may be disposed between the expandable framework and native tissue in order to prevent fluid, such as blood, flowing around the expandable framework in a downstream direction so as to ensure that the plurality of valve leaflets can stop the flow of fluid when in the closed position.

In some embodiments, the plurality of valve leaflets may be comprised of a polymer, such as a thermoplastic polymer. In some embodiments, the plurality of valve leaflets may include at least 50 percent by weight of a polymer. In some embodiments, the plurality of valve leaflets may be formed from bovine pericardial or other living tissue. Other configurations and/or materials are also contemplated.

In some embodiments, the inner skirt and/or the outer skirt may include a polymer, such as a thermoplastic polymer. In some embodiments, the inner skirt and/or the outer skirt may include at least 50 percent by weight of a polymer. In some embodiments one or more of the plurality of valve leaflets, the inner skirt, and/or the outer skirt may be formed of the same polymer or polymers. In some embodiments, the polymer may be a polyurethane. In some embodiments, the inner skirt and/or the outer skirt may be substantially impervious to fluid. In some embodiments, the inner skirt and/or the outer skirt may be formed from a thin tissue (e.g., bovine pericardial, etc.). In some embodiments, the inner skirt and/or the outer skirt may be formed from a coated fabric material. In some embodiments, the inner skirt and/or the outer skirt may be formed from a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the replacement heart valve implant 50 and/or the expandable framework may have an outer extent of about 23 millimeters (mm), about 25 mm, about 27 mm, about 30 mm, etc. in an unconstrained configuration (e.g., in the expanded configuration). In some embodiments, the replacement heart valve implant 50 and/or the expandable framework may have an outer extent of about 10 mm, about 9 mm about 8 mm, about 7 mm, about 6 mm, etc. in the collapsed configuration. Other configurations are also contemplated.

In some embodiments, the system 100 may be configured to permit delivery of the replacement heart valve implant 50 to the treatment site while the heart remains beating, for example, using a minimally invasive surgical and/or percutaneous procedure. In some embodiments, the system 100 may be configured for introduction into the anatomical vascular system, and for advancement along the vascular system to the treatment site. In some embodiments, the system 100 may be configured for introduction into the femoral artery, and guided retrograde via the descending aorta, aortic arch, and ascending aorta to the heart (sometimes called transfemoral access). In some embodiments, the system 100 may be insertable via the subclavian artery and guided retrograde to the heart (sometimes called transubclavian access). In some embodiments, the system 100 may be inserted directly into a chamber of the heart such as a ventricle (for example, left ventricle) via a direct access route while the heart remains beating. For example, a direct access route may be through an aperture opened in the apex of the heart (sometimes called transapical access). Other configurations are also contemplated.

It may be appreciated that during delivery and/or deployment of the replacement heart valve implant 50, portions of the system 100 may be required to be advanced through tortuous and/or narrow body lumens. Therefore, it may be desirable to utilize components and/or to design configurations that reduce the profile of portions of the systems while maintaining sufficient strength (e.g., compressive, torsional, etc.) and flexibility of the systems as a whole.

In some embodiments, an introducer sheath may be inserted into the patient's anatomy to gain access to the vascular system. In some embodiments, the introducer sheath may include a valve or other means of preventing fluid backflow out of the introducer sheath. At least a portion of the system 100 may be inserted into and/or through the introducer sheath and into the vascular system for advancement to the treatment site.

In some embodiments, the system 100 for delivering the replacement heart valve implant 50 may include a proximal handle 110 and a valve capsule 120 spaced apart from the proximal handle 110. FIGS. 1 and 2 illustrate the valve capsule 120 in a delivery configuration. In the delivery configuration, the valve capsule 120 may be configured to receive and/or retain the replacement heart valve implant 50 in the collapsed configuration, as seen in FIG. 2 for example. The valve capsule 120 may be configured to cover at least a portion of the replacement heart valve implant 50 during delivery of the replacement heart valve implant 50 to the treatment site.

The system 100 may include an inner shaft 130 extending distally from the proximal handle 110 to the valve capsule 120. The system 100 may include an outer sheath 140 coaxially disposed over the inner shaft 130 and extending distally from the proximal handle 110 to the valve capsule 120. The system 100 may include a positioning sheath 150 coaxially disposed over the outer sheath 140 and extending distally from the proximal handle 110 to a distal end 152 spaced apart proximally from the valve capsule 120. In some embodiments, the inner shaft 130, the outer sheath 140, and/or the positioning sheath 150 may be movable relative to each other, as discussed herein.

In some embodiments, the valve capsule 120 may include a proximal capsule portion 122 and a distal capsule portion 124. In some embodiments, the proximal capsule portion 122 may open toward the distal capsule portion 124, and/or the distal capsule portion 124 may open toward the proximal capsule portion 122. For example, the proximal capsule portion 122 may open in a distal direction and the distal capsule portion 124 may open in a proximal direction.

In some embodiments, the proximal capsule portion 122 may have a length greater than a length of the distal capsule portion 124. For example, the ratio of the length of the proximal capsule portion 122 divided by the length of the distal capsule portion 124 may be at least 1.1, optionally at least 1.2, optionally at least 1.3, optionally at least 1.4, optionally at least 1.5, optionally at least 1.6, optionally at least 1.7, optionally at least 1.8, optionally at least 1.9, optionally at least 2.0, optionally at least 2.1, optionally at least 2.2, optionally at least 2.3, optionally at least 2.4, optionally at least 2.5, optionally at least 2.6, optionally at least 2.7, optionally at least 2.8, optionally at least 2.9, optionally at least 3, optionally at least 3.5, optionally at least 4 or optionally at least 4.5, or optionally at least 5.

The proximal capsule portion 122 may be configured to cover a first portion of the replacement heart valve implant 50 and the distal capsule portion 124 may be configured to cover a second portion of the replacement heart valve implant 50 for percutaneous delivery of the replacement heart valve implant 50 to the treatment site. The first portion of the replacement heart valve implant 50 may be different from the second portion of the replacement heart valve implant 50. The inner shaft 130 may extend longitudinally and/or axially through the replacement heart valve implant 50 when the replacement heart valve implant 50 is disposed within the valve capsule 120. In at least some embodiments, the inner shaft 130 may include a guidewire lumen extending therethrough.

The proximal capsule portion 122 may be fixedly attached to a distal portion of the outer sheath 140. The distal capsule portion 124 may be fixedly attached to a distal portion of the inner shaft 130. In at least some embodiments, the proximal capsule portion 122 may be spaced apart longitudinally and/or axially from the distal capsule portion 124. As such, there may be no overlap between the proximal capsule portion 122 and the distal capsule portion 124 (e.g., the proximal capsule portion 122 does not longitudinally and/or axially overlap any part of the distal capsule portion 124). In some embodiments, the proximal capsule portion 122 may be spaced apart longitudinally and/or axially from the distal capsule portion 124 by a first distance in the delivery configuration. In some embodiments, the first distance may be about 4 mm in the delivery configuration. In some embodiments, the first distance may be about 5 mm in the delivery configuration. In some embodiments, the first distance may be about 6 mm in the delivery configuration. Other configurations are also contemplated.

The proximal capsule portion 122 and the distal capsule portion 124 may be longitudinally and/or axially translatable relative to each other between the delivery configuration (e.g., FIG. 2) and a deployment configuration (e.g., FIG. 4). In some embodiments, the proximal capsule portion 122 and the outer sheath 140 may be longitudinally and/or axially translatable relative to the distal capsule portion 124 and the inner shaft 130 in opposite directions between the delivery configuration (e.g., FIG. 2) and the deployment configuration (e.g., FIG. 4). For example, the proximal capsule portion 122 and the outer sheath 140 may be translatable in a proximal direction and the distal capsule portion 124 and the inner shaft 130 may be translatable in a distal direction. Other configurations are also contemplated.

In some embodiments, the proximal capsule portion 122 may be spaced apart longitudinally and/or axially from the distal capsule portion 124 by a second distance in the deployment configuration (e.g., FIG. 4). The second distance may be greater than the first distance. In some embodiments, the second distance may be about 40 mm in the deployment configuration. In some embodiments, the second distance may be about 44 mm in the deployment configuration. In some embodiments, the second distance may be about 45 mm in the deployment configuration. In some embodiments, the second distance may be about 46 mm in the deployment configuration. In some embodiments, the second distance may be about 50 mm in the deployment configuration. Other configurations are also contemplated.

In the deployment configuration, the valve capsule 120 may be configured to release the replacement heart valve implant 50 such that the replacement heart valve implant 50 may expand from the compressed configuration to the expanded configuration. In at least some embodiments, the replacement heart valve implant 50 may be released and/or deployed at and/or within the treatment site (e.g., the native aortic valve, etc.).

As may be seen in FIGS. 2-4, the system 100 may include a guide tube 160 disposed within the proximal handle 110. In at least some embodiments, the guide tube 160 may be formed from a metallic material. In some embodiments, the guide tube 160 may be formed from a polymeric material. the guide tube 160 may be formed from a composite material. Other configurations, including combinations thereof, are also contemplated. The guide tube 160 may include a lumen extending therein. The guide tube 160 may include a distal longitudinal slot 162 formed through a wall 164 of the guide tube 160 in a distal portion of the guide tube 160. The guide tube 160 may include a proximal longitudinal slot 163 formed through a wall 164 of the guide tube 160 in a proximal portion of the guide tube 160.

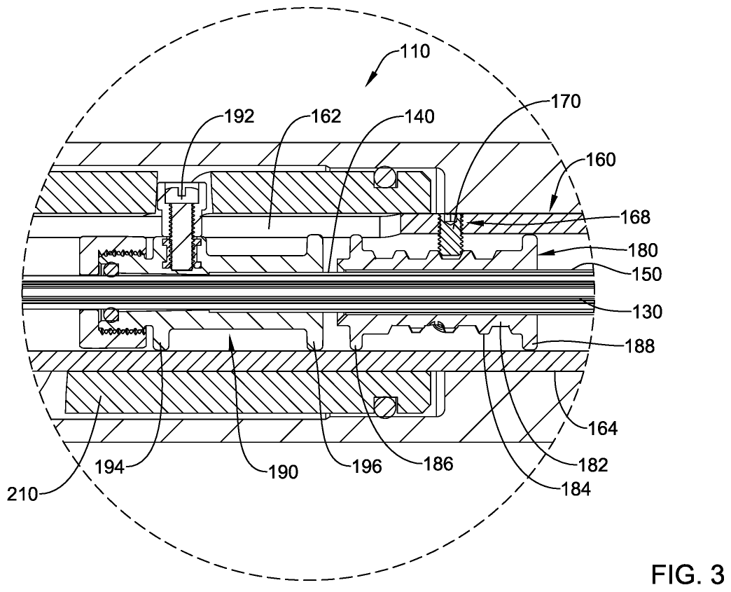

In some embodiments, the guide tube 160 includes at least one set screw 170 threadably engaged with the wall 164 of the guide tube 160 in the distal portion of the guide tube 160 and extending radially inward therefrom, as seen in FIG. 3. The guide tube 160 may include at least one threaded aperture 168 formed in the wall 164 of the guide tube 160 to threadably receive and/or engage the at least one set screw 170. In some embodiments, the at least one set screw 170 may include two or more set screws. For example, the at least one set screw 170 may include two set screws, three set screws, four set screws, etc.

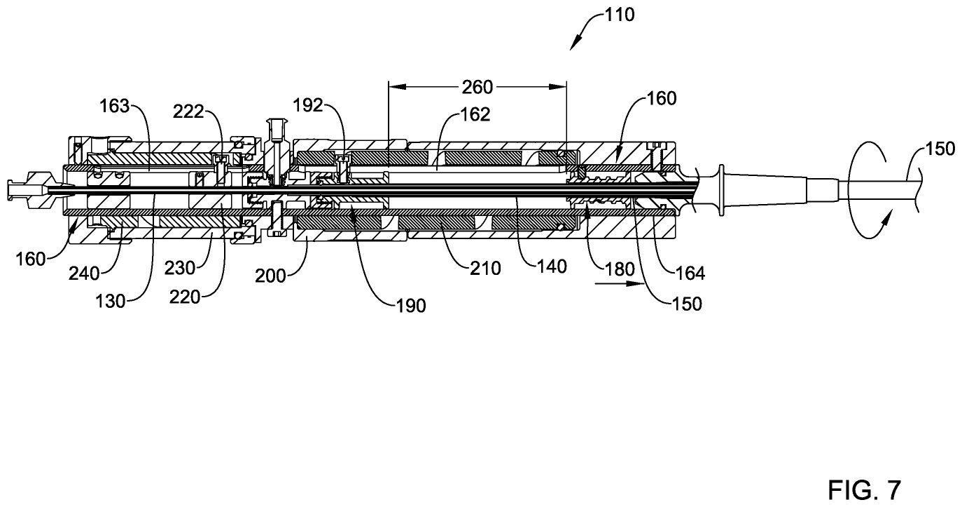

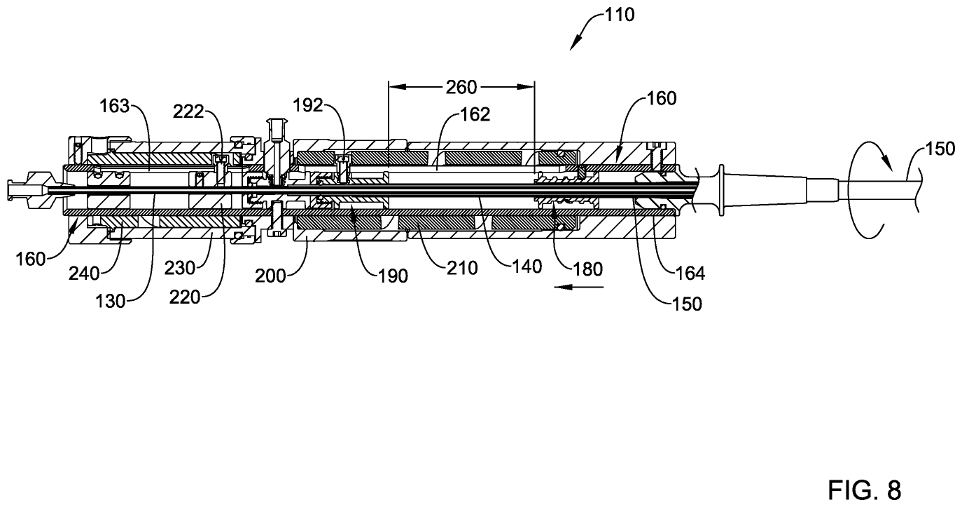

The system 100 may include a distal hub 180 fixedly attached to a proximal end of the positioning sheath 150. The distal hub 180 may be disposed within and selectively movable relative to the guide tube 160 via rotation of the positioning sheath 150 (e.g., FIGS. 7-8). In at least some embodiments, the distal hub 180 may be disposed within the lumen of the guide tube 160 within the distal portion of the guide tube 160. The distal hub 180 may be disposed coaxially over the inner shaft 130 and/or the outer sheath 140. The distal hub 180 may be disposed coaxially and/or concentrically within the guide tube 160.

The distal hub 180 may include a body portion 182 and a helical ridge 184 extending radially outward from the body portion 182, as shown in FIG. 3. In some embodiments, the distal hub 180 may include a proximal flange 186 disposed proximate a proximal end of the distal hub 180 and/or a distal flange 188 disposed proximate a distal end of the distal hub 180. In some embodiments, the proximal flange 186 and/or the distal flange 188 may be configured to engage and/or slide along the wall 164 of the guide tube 160. In some embodiments, the proximal flange 186 may have a radial extent less than an inner diameter of the lumen of the guide tube 160. In some embodiments, the distal flange 188 may have a radial extent less than the inner diameter of the lumen of the guide tube 160. In at least some embodiments, the proximal flange 186 may extend radially outward from the body portion 182 farther than the helical ridge 184. In at least some embodiments, the distal flange 188 may extend radially outward from the body portion 182 farther than the helical ridge 184.

In some embodiments, the distal hub 180 may be formed from a polymeric material. In some embodiments, the distal hub 180 may be formed from a composite material. In some embodiments, the distal hub 180 may be formed from a metallic material. Other configurations, including combinations thereof, are also contemplated. In some embodiments, the distal hub 180 may be overmolded onto the proximal end of the positioning sheath 150. In some embodiments, the distal hub 180 may be formed separately from the positioning sheath 150 and later fixedly attached to the proximal end of the positioning sheath 150, such as by adhesive bonding, welding, friction and/or interference fit, mechanical attachment, etc.

In some embodiments, the system 100 may include a proximal hub 190 fixedly attached to a proximal end of the outer sheath 140. The proximal hub 190 may be disposed within and selectively movable axially relative to the guide tube 160. In at least some embodiments, the proximal hub 190 may be disposed within the lumen of the guide tube 160. The proximal hub 190 may be disposed within the distal portion of the guide tube 160 proximal of the distal hub 180.

The proximal hub 190 may include a guide member 192 extending radially outward from the proximal hub 190. In some embodiments, the proximal hub 190 may include a proximal flange 194 disposed proximate a proximal end of the proximal hub 190 and/or a distal flange 196 disposed proximate a distal end of the proximal hub 190. In some embodiments, the proximal flange 194 and/or the distal flange 196 may be configured to engage and/or slide along the wall 164 of the guide tube 160. In some embodiments, the proximal flange 194 may have a radial extent less than an inner diameter of the lumen of the guide tube 160. In some embodiments, the distal flange 196 may have a radial extent less than the inner diameter of the lumen of the guide tube 160.

In some embodiments, the proximal hub 190 may be formed from a polymeric material. In some embodiments, the proximal hub 190 may be formed from a composite material. In some embodiments, the proximal hub 190 may be formed from a metallic material. Other configurations, including combinations thereof, are also contemplated. In some embodiments, the proximal hub 190 may be overmolded onto the proximal end of the outer sheath 140. In some embodiments, the proximal hub 190 may be formed separately from the outer sheath 140 and later fixedly attached to the proximal end of the outer sheath 140, such as by adhesive bonding, welding, friction and/or interference fit, mechanical attachment, etc.

The system 100 and/or the proximal handle 110 may include a first collar 200 rotatably disposed around the distal portion of the guide tube 160, as seen in FIGS. 2 and 4. In some embodiments, rotation of the first collar 200 about the distal portion of the guide tube 160 may be configured to move the proximal hub 190 axially within the guide tube 160.

The system 100 and/or the proximal handle 110 may include a first helical guide 210 disposed radially outward of the distal portion of the guide tube 160 and radially inward of the first collar 200. The first helical guide 210 may be configured to rotate about the distal portion of the guide tube 160. In some embodiments, the first collar 200 may be nonrotatably engaged with the first helical guide 210 such that rotation of the first collar 200 rotates the first helical guide 210. In some embodiments, at least a portion of the guide member 192 may be disposed within the distal longitudinal slot 162 of the guide tube 160. The guide member 192 may also extend into the first helical guide 210.

In use, as the first collar 200 is rotated about the distal portion of the guide tube 160, the first helical guide 210 may also rotate about the distal portion of the guide tube 160, thereby urging and/or moving the guide member 192 and/or the proximal hub 190 axially along the distal longitudinal slot 162. Consequently, the outer sheath 140 may move longitudinally and/or axially as the proximal hub 190 moves longitudinally and/or axially within the distal portion of the guide tube 160, thereby causing the proximal capsule portion 122 to move longitudinally and/or axially between the delivery configuration and the deployment configuration.

In some embodiments, the system 100 and/or the proximal handle 110 may include a slide block 220 slidably disposed within the proximal portion of the guide tube 160. The inner shaft 130 may extend longitudinally and/or axially through the slide block 220. In at least some embodiments, the slide block 220 may be fixedly secured to the inner shaft 130. In some embodiments, the slide block 220 may be fixedly secured to the inner shaft 130 using a locking element 222 such as a set screw, a pin, etc. In some embodiments, the system 100 and/or the proximal handle 110 may include a second collar 230 rotatably disposed around the proximal portion of the guide tube 160. In some embodiments, rotation of the second collar 230 about the proximal portion of the guide tube 160 may be configured to move the inner shaft 130 axially within and/or relative to the outer sheath 140, the positioning sheath 150, and/or the guide tube 160.

The system 100 and/or the proximal handle 110 may include a second helical guide 240 disposed radially outward of the proximal portion of the guide tube 160 and radially inward of the second collar 230. The second helical guide 240 may be configured to rotate about the proximal portion of the guide tube 160. In some embodiments, the second collar 230 may be nonrotatably engaged with the second helical guide 240 such that rotation of the second collar 230 rotates the second helical guide 240. The locking element 222 may extend radially outward from the slide block 220. In some embodiments, at least a portion of the locking element 222 may be disposed within the proximal longitudinal slot 163 of the guide tube 160. The locking element 222 may also extend into the second helical guide 240.

In use, as the second collar 230 is rotated about the proximal portion of the guide tube 160, the second helical guide 240 may also rotate about the proximal portion of the guide tube 160, thereby urging and/or moving the locking element 222 and/or the slide block 220 axially along the proximal longitudinal slot 163. Consequently, the inner shaft 130 may move longitudinally and/or axially as the slide block 220 moves longitudinally and/or axially within the proximal portion of the guide tube 160, thereby causing the distal capsule portion 124 to move longitudinally and/or axially between the delivery configuration and the deployment configuration.

As such, during use, the first collar 200 and the second collar 230 may be used together, or in some alternative configurations separately, to release and/or deploy the replacement heart valve implant 50 from the valve capsule 120 at the treatment site by moving the proximal capsule portion 122 and the distal capsule portion 124 apart from each other toward and/or to the deployment configuration.

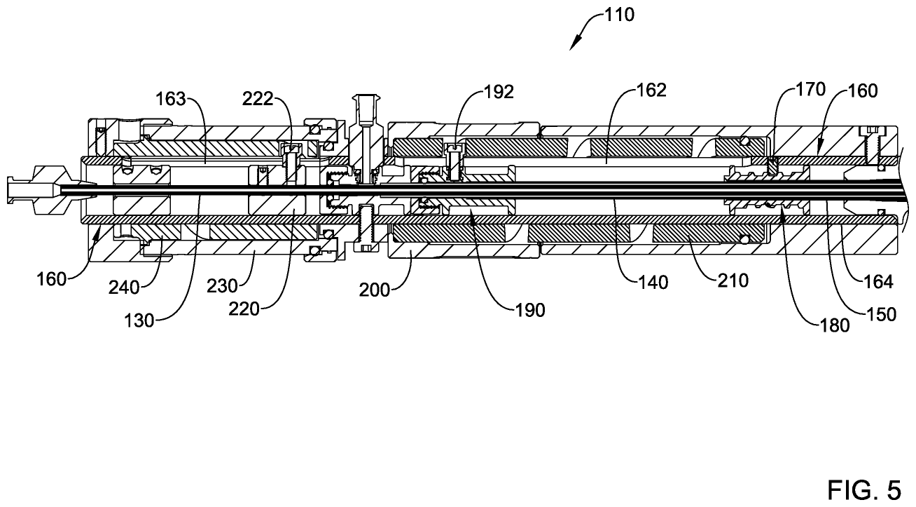

In some embodiments, a method of manufacturing and/or assembling the system 100 for delivering the replacement heart valve implant 50 may include positioning the proximal hub 190 and the distal hub 180 within the guide tube 160 of the proximal handle 110 of the system 100, as seen in FIGS. 4-6. In some embodiments, the at least one set screw 170 may be configured to extend between adjacent turns of the helical ridge 184 of the distal hub 180, as seen in FIGS. 2-5 for example. In at least some embodiments, the at least one set screw 170 may be disengaged from the body portion 182 of the distal hub 180 (e.g., spaced apart from and/or not in contact with the body portion 182).

The method of manufacturing and/or assembling the system 100 for delivering the replacement heart valve implant 50 may include setting a first predetermined distance 250 (e.g., FIG. 4) between the proximal capsule portion 122 and the distal capsule portion 124 of the valve capsule 120. The first predetermined distance 250 may correspond to the second distance in the deployment configuration discussed above. In at least some embodiments, the second distance is the first predetermined distance 250.

Setting the first predetermined distance 250 may include moving the outer sheath 140 longitudinally and/or axially relative to the inner shaft 130. In some embodiments, moving the outer sheath 140 longitudinally and/or axially relative to the inner shaft 130 may include moving the proximal hub 190 proximally and/or distally within the guide tube 160. After setting the first predetermined distance 250, the proximal capsule portion 122 and the distal capsule portion 124 of the valve capsule 120 may be held in a fixed position relative to each other. In some embodiments, the proximal capsule portion 122 and the distal capsule portion 124 of the valve capsule 120 may be held in the fixed position relative to each other with a fixture. In some embodiments, a user or technician assembling the system 100 may hold the proximal capsule portion 122 and the distal capsule portion 124 of the valve capsule 120 may be held in the fixed position relative to each other. Other configurations, including combinations thereof, are also contemplated.

In some embodiments, the first predetermined distance 250 may be about 40 mm. In some embodiments, the first predetermined distance 250 may be about 42 mm. In some embodiments, the first predetermined distance 250 may be about 44 mm. In some embodiments, the first predetermined distance 250 may be about 46 mm. In some embodiments, the first predetermined distance 250 may be about 48 mm. In some embodiments, the first predetermined distance 250 may be about 50 mm. Other dimensions and/or values are also contemplated.

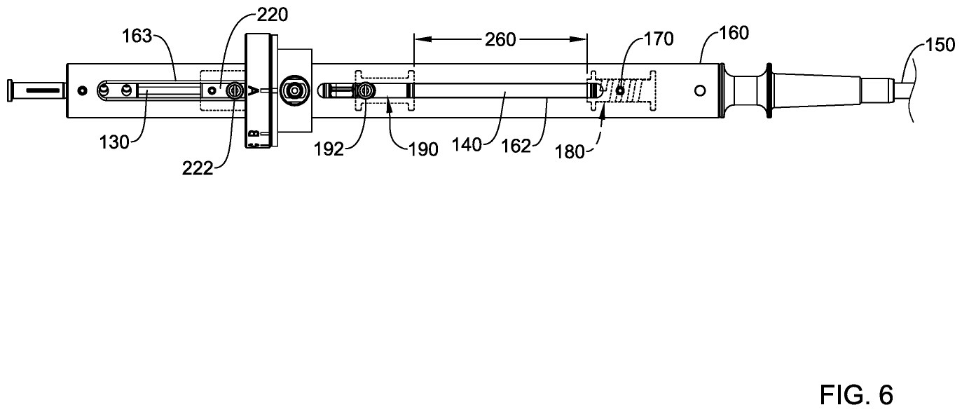

In some embodiments, the method of manufacturing and/or assembling the system 100 for delivering the replacement heart valve implant 50 may include axially moving the positioning sheath 150 relative to the outer sheath 140 to set a second predetermined distance 260 (e.g., FIG. 6) between the proximal hub 190 and the distal hub 180. In some embodiments, axially moving the positioning sheath 150 relative to the outer sheath 140 may include rotating the positioning sheath 150 relative to the outer sheath 140, as seen in FIGS. 7-8. As discussed herein, the at least one set screw 170 may be configured to extend between adjacent turns of the helical ridge 184 of the distal hub 180. As such, when the at least one set screw 170 extends between adjacent turns of the helical ridge 184 of the distal hub 180, rotation of the positioning sheath 150 relative to the outer sheath 140 may also rotate the distal hub 180 relative to the at least one set screw 170 and/or the outer sheath 140 to move the distal hub 180 longitudinally and/or axially relative to the guide tube 160 when the at least one set screw 170 is disengaged from the body portion 182 of the distal hub 180.

As the positioning sheath 150 and/or the distal hub 180 is rotated, the distal hub 180 may be advanced distally and/or withdrawn proximally relative to the at least one set screw 170 and/or the outer sheath 140. For example, clockwise rotation of the positioning sheath 150, as viewed proximally to distally, may rotate the distal hub clockwise and advance the distal hub 180 distally within the guide tube 160 and/or relative to the at least one set screw 170 and/or the outer sheath 140, as shown in FIG. 7. Similarly, counterclockwise rotation of the positioning sheath 150, as viewed proximally to distally, may rotate the distal hub counterclockwise and withdraw the distal hub 180 proximally within the guide tube 160 and/or relative to the at least one set screw 170 and/or the outer sheath 140, as shown in FIG. 8. Other configurations, including configurations opposite of those examples explicitly described above, are also contemplated.

In some embodiments, mechanical interference between the at least one set screw 170 and the helical ridge 184 may prevent longitudinal and/or axial movement of the distal hub 180 relative to the guide tube 160 when an axial force is applied to the distal hub 180 while the at least one set screw 170 extends between adjacent turns of the helical ridge 184 of the distal hub 180 the at least one set screw 170 extends between adjacent turns of the helical ridge 184 of the distal hub 180. For example, applying only longitudinal and/or axial force to the positioning sheath 150 and/or the distal hub 180 may be insufficient to move the distal hub 180 longitudinally and/or axially relative to the guide tube 160 when the at least one set screw 170 extends between adjacent turns of the helical ridge 184 of the distal hub 180. Rotation of the positioning sheath 150 and the distal hub 180 fixedly attached thereto is required to cause longitudinal and/or axial movement of the distal hub 180 relative to the guide tube 160 when the at least one set screw 170 extends between adjacent turns of the helical ridge 184 of the distal hub 180.

In some embodiments, the second predetermined distance 260 may be about 40 mm. In some embodiments, the second predetermined distance 260 may be about 41 mm. In some embodiments, the second predetermined distance 260 may be about 42 mm. In some embodiments, the second predetermined distance 260 may be about 43 mm. In some embodiments, the second predetermined distance 260 may be about 44 mm. In some embodiments, the second predetermined distance 260 may be about 45 mm. Other dimensions and/or values are also contemplated. In at least some embodiments, the second predetermined distance 260 may be less than the first predetermined distance 250.

In some embodiments, the at least one set screw 170 may be configured to engage with the body portion 182 of the distal hub 180, as seen in FIGS. 7-8 for example. In some embodiments, the method may include engaging the at least one set screw 170 with the body portion 182 of the distal hub 180 to secure the distal hub 180 longitudinally and/or axially within the guide tube 160 at the second predetermined distance 260 from the proximal hub 190. Engagement of the at least one set screw 170 with the body portion 182 of the distal hub 180 may substantially prevent rotation of the distal hub 180 relative to the at least one set screw 170 and/or the guide tube 160. Similarly, engagement of the at least one set screw 170 with the body portion 182 of the distal hub 180 may prevent longitudinal and/or axial movement of the distal hub 180 relative to the at least one set screw 170 and/or the guide tube 160 due to the aforementioned mechanical interference between the at least one set screw 170 and the helical ridge 184 of the distal hub 180.

In some embodiments, the method of manufacturing and/or assembling the system 100 for delivering the replacement heart valve implant 50 may include attaching the first helical guide 210 and/or the first collar 200 to the proximal handle 110 over the distal portion of the guide tube 160. In some embodiments, the method of manufacturing and/or assembling the system 100 for delivering the replacement heart valve implant 50 may include attaching the second helical guide 240 and/or the second collar 230 to the proximal handle 110 over the proximal portion of the guide tube 160. In some embodiments, the method of manufacturing and/or assembling the system 100 for delivering the replacement heart valve implant 50 may include attaching a handle shell over the first helical guide 210 distal of the first collar 200. The first helical guide 210 may be configured to rotate within the handle shell as the first collar 200 is rotated relative to the handle shell and/or the guide tube 160.

In use, the system 100 may be used to deliver the replacement heart valve implant 50 to the treatment site. As discussed herein, the replacement heart valve implant 50, in the collapsed configuration, may be disposed within the valve capsule 120 in the delivery configuration, shown in FIGS. 1-2. The proximal capsule portion 122 may be spaced apart longitudinally and/or axially from the distal capsule portion 124 by the first distance in the delivery configuration (e.g., FIGS. 1-2). At the treatment site, the proximal handle 110 may be used, actuated, and/or manipulated to shift the valve capsule 120 to the deployment configuration to release the replacement heart valve implant 50, thereby permitting the replacement heart valve implant 50 to shift from the collapsed configuration (e.g., FIGS. 1-2) to the expanded configuration (e.g., FIG. 4). In the deployment configuration (e.g., FIG. 4), the proximal capsule portion 122 may be spaced apart longitudinally and/or axially from the distal capsule portion 124 by the second distance (e.g., the first predetermined distance 250).

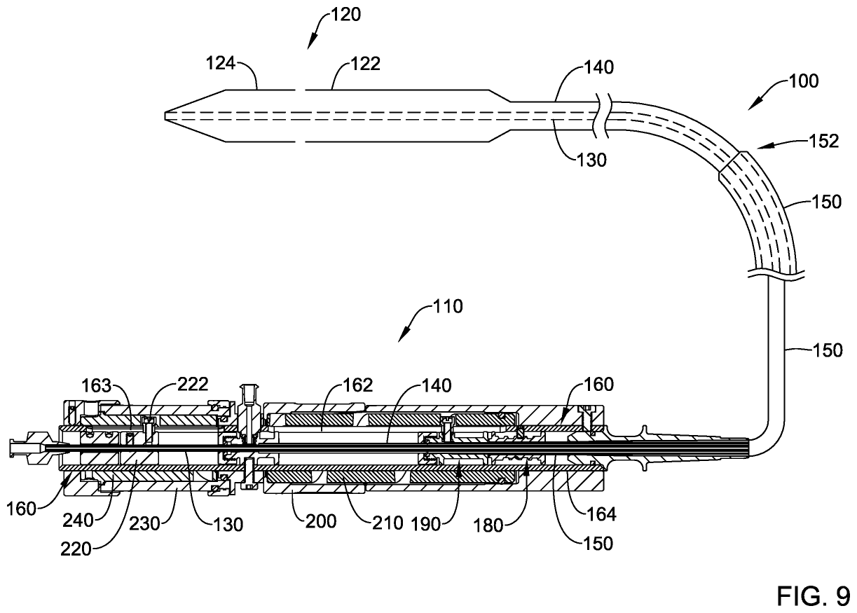

After deploying the replacement heart valve implant 50, the proximal handle 110 may be used, actuated, and/or manipulated to shift the valve capsule 120 toward and/or to the withdrawal configuration, seen in FIG. 9. After deploying the replacement heart valve implant 50, distal longitudinal and/or axial movement of the proximal hub 190 within and/or relative to the guide tube 160 may bring the proximal hub 190 into contact with the distal hub 180 and apply axial force to the distal hub 180 in a distal direction. Distal longitudinal and/or axial movement of the proximal hub 190 may be accomplished and/or provided by rotation of the first collar 200 and/or the first helical guide 210, thereby urging and/or driving the guide member 192 of the proximal hub 190 distally within the distal longitudinal slot 162. The distal hub 180 may function as a hard stop for longitudinal and/or axial movement of the proximal hub 190 in the distal direction, thereby positioning the proximal capsule portion 122 and the distal capsule portion 124 spaced apart by a third distance in the withdrawal configuration (e.g., FIG. 9). The third distance may be less than the first distance.

In some embodiments, the third distance may be about 2 mm. In some embodiments, the third distance may be about 2.5 mm. In some embodiments, the third distance may be about 3 mm. In some embodiments, the third distance may be about 3.5 mm. In some embodiments, the third distance may be about 4 mm. Other configurations and/or values are also contemplated.

Since the third distance is less than the first distance, there may be less of a gap between the proximal capsule portion 122 and the distal capsule portion 124 to catch on adjacent patient anatomy, other medical devices, etc. However, it may be important and/or beneficial to avoid contact between the proximal capsule portion 122 and the distal capsule portion 124. Accordingly, the distal hub 180, acting as the hard stop for the proximal hub 190, prevents the proximal capsule portion 122 from colliding with, running into, and/or axially overlapping the distal capsule portion 124 as the valve capsule 120 is shifted to the withdrawal configuration, thereby preventing damage to the valve capsule 120 and/or injury to the patient that may result from said damage during withdrawal of the system 100.

As the valve capsule 120 is shifted from the deployment configuration toward and/or to the withdrawal configuration, the proximal handle 110 and/or the first collar 200 (in conjunction with the first helical guide 210) may provide relatively easy movement of the proximal hub 190 in the distal direction when the replacement heart valve implant 50 is not present within the valve capsule 120. In order to reduce practitioner training requirements and/or improve consistency of results, the use of the distal hub 180 as a hard stop for the proximal hub 190 when closing the valve capsule 120 (e.g., shifting the valve capsule 120 from the deployment configuration to the withdrawal configuration) cannot move or shift longitudinally and/or axially during use. The mechanical interference between the at least one set screw 170 and the helical ridge 184 prevents longitudinal and/or axial movement of the distal hub 180, thereby enhancing the function of the distal hub 180 as a hard stop for the proximal hub 190. However, assembly of the system 100 during manufacturing requires at least some adjustability to be built into the system 100 to account for tolerances, etc. The same combination of features (e.g., the at least one set screw 170 and the helical ridge 184, and the mechanical interference between them) that functions to prevent longitudinal and/or axial movement of the distal hub 180 also permits adjustability of the positioning of the distal hub 180 via rotation of the positioning sheath 150 and/or the distal hub 180 to cause longitudinal and/or axial movement of the distal hub 180 relative to the guide tube 160 when the at least one set screw 170 is disengaged from the body portion 182 of the distal hub 180.

The materials that can be used for the various components of the device and the various elements thereof disclosed herein may include those commonly associated with medical devices and devices used and/or associated with medical devices. For simplicity purposes, the following discussion refers to the system. However, this is not intended to limit the devices and methods described herein, as the discussion may be applied to other elements, members, components, or devices disclosed herein, such as, but not limited to, the replacement heart valve implant, the proximal handle, the valve capsule, the inner shaft, the outer sheath, the positioning sheath, the guide tube, the proximal hub, the distal hub, the at least one set screw, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® or ChronoSil®), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system may include a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. In some embodiments, the yarns may be made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible system.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for delivering a replacement heart valve implant, comprising:
a proximal handle and a valve capsule spaced apart from the proximal handle, the valve capsule being configured to receive the replacement heart valve implant;
an inner shaft extending distally from the proximal handle to the valve capsule;
an outer sheath coaxially disposed over the inner shaft and extending distally from the proximal handle to the valve capsule;
a positioning sheath coaxially disposed over the outer sheath and extending distally from the proximal handle to a distal end spaced apart proximally from the valve capsule;
a guide tube disposed within the proximal handle; and
a distal hub fixedly attached to a proximal end of the positioning sheath, the distal hub being disposed within and selectively movable relative to the guide tube via rotation of the positioning sheath.

2. The system of claim 1, wherein the distal hub includes a body portion and a helical ridge extending radially outward from the body portion.

3. The system of claim 2, wherein the guide tube includes at least one set screw threadably engaged with a wall of the guide tube and configured to extend between adjacent turns of the helical ridge.

4. The system of claim 3, wherein the at least one set screw includes two or more set screws.

5. The system of claim 1, further comprising a proximal hub fixedly attached to a proximal end of the outer sheath, the proximal hub being disposed within and selectively movable axially relative to the guide tube.

6. The system of claim 5, wherein the proximal handle includes a first collar rotatably disposed around the guide tube;
wherein rotation of the first collar about the guide tube is configured to move the proximal hub axially within the guide tube.

7. The system of claim 5, wherein the distal hub acts as a hard stop for axial movement of the proximal hub in a distal direction.

8. The system of claim 1, wherein the valve capsule includes a proximal capsule portion fixedly attached to a distal portion of the outer sheath and a distal capsule portion fixedly attached to a distal portion of the inner shaft.

9. The system of claim 8, wherein the proximal capsule portion is configured to cover a first portion of the replacement heart valve implant and the distal capsule portion is configured to cover a second portion of the replacement heart valve implant for percutaneous delivery of the replacement heart valve implant to a treatment site.

10. A method of manufacturing a system for delivering a replacement heart valve implant, comprising:
positioning a proximal hub and a distal hub within a guide tube of a proximal handle of the system, wherein:
an inner shaft extends through the guide tube to a distal capsule portion of a valve capsule spaced apart from the proximal handle, the valve capsule being configured to receive the replacement heart valve implant;
the proximal hub is fixedly attached to a proximal end of an outer sheath coaxially disposed over the inner shaft and extending distally from the proximal handle to a proximal capsule portion of the valve capsule;
the distal hub is fixedly attached to a proximal end of a positioning sheath coaxially disposed over the outer sheath and extending distally from the proximal handle to a distal end spaced apart proximally from the valve capsule; and
at least one set screw is threadably engaged with a wall of the guide tube;
setting a first predetermined distance between the proximal capsule portion and the distal capsule portion;
axially moving the positioning sheath relative to the outer sheath to set a second predetermined distance between the proximal hub and the distal hub; and
engaging the at least one set screw with a body portion of the distal hub to secure the distal hub axially within the guide tube at the second predetermined distance from the proximal hub.

11. The method of claim 10, wherein axially moving the positioning sheath relative to the outer sheath includes rotating the positioning sheath relative to the outer sheath.

12. The method of claim 10, wherein the distal hub includes a helical ridge extending radially outward from the body portion and the at least one set screw extends between adjacent turns of the helical ridge.

13. The method of claim 12, wherein engagement of the at least one set screw with the body portion of the distal hub prevents axial movement of the distal hub relative to the guide tube.

14. The method of claim 12, wherein the distal hub includes a proximal flange extending radially outward from the body portion farther than the helical ridge and a distal flange extending radially outward from the body portion farther than the helical ridge.

15. The method of claim 10, wherein the second predetermined distance is less than the first predetermined distance.

16. A system for delivering a replacement heart valve implant configured to shift between a collapsed configuration and an expanded configuration, the system comprising:
a proximal handle and a valve capsule spaced apart from the proximal handle, the valve capsule being configured to receive and retain the replacement heart valve implant in the collapsed configuration;
an inner shaft extending distally from the proximal handle to the valve capsule, wherein the inner shaft extends axially through the replacement heart valve implant when the replacement heart valve implant is disposed within the valve capsule;
an outer sheath coaxially disposed over the inner shaft and extending distally from the proximal handle to the valve capsule;
a positioning sheath coaxially disposed over the outer sheath and extending distally from the proximal handle to a distal end spaced apart proximally from the valve capsule;
a guide tube disposed within the proximal handle; and
a distal hub fixedly attached to a proximal end of the positioning sheath and disposed coaxially over the inner shaft, the distal hub being disposed within and selectively movable axially relative to the guide tube.

17. The system of claim 16, wherein the guide tube is formed from a metallic material.

18. The system of claim 16, wherein the distal hub is formed from a polymeric material.

19. The system of claim 16, wherein the distal hub includes a body portion and a helical ridge extending radially outward from the body portion;
wherein the guide tube includes at least one set screw threadably engaged with a wall of the guide tube and extending radially inward therefrom;
wherein when the at least one set screw extends between adjacent turns of the helical ridge:
rotation of the positioning sheath moves the distal hub axially relative to the guide tube when the at least one set screw is disengaged from the body portion of the distal hub; and
mechanical interference between the at least one set screw and the helical ridge prevents axial movement of the distal hub relative to the guide tube when an axial force is applied to the distal hub.

20. The system of claim 19, further comprising a proximal hub fixedly attached to a proximal end of the outer sheath, the proximal hub being disposed within and selectively movable axially relative to the guide tube;
wherein distal movement of the proximal hub within the guide tube brings the proximal hub into contact with the distal hub and applies the axial force to the distal hub in a distal direction.
